(19) United States
(12) Patent Application Publication
(10) Pub. No.: US 2022/0389511 A1
(43) Pub. Date: Dec. 8, 2022

(54) SYSTEMS AND METHODS FOR ARTIFICAL INTELLIGENCE BASED CELL ANALYSIS

(71) Applicant: Aikili Biosystems, Inc., Allston, MA (US)

(72) Inventor: Laura E. KELLEY, Cambridge, MA (US)

(21) Appl. No.: 17/642,030

(22) PCT Filed: Sep. 14, 2020
(86) PCT No.: PCT/US2020/050643

§ 371 Date: Mar. 10, 2022

Related U.S. Application Data
(60) Provisional application No. 62/899,993, filed on Sep. 13, 2019.

(51) Int. Cl. C12Q 1/6886; G06V 10/84; G06N 3/04; G06T 7/11; G06T 7/00

(52) U.S. Cl. CPC C12Q 1/6886; G06V 10/84; G06N 3/0454; G06T 7/11; G06T 7/0012; G06T 2207/20084; G06T 2207/30024; C12Q 2600/158

(57) ABSTRACT

Diagnostic and prognostic assays and a portable point of care system is provided that performs such assays using an automated, artificial intelligence (AI) based molecular analyses of a subject sample. The system provides for rapid, cost-efficient multiplexable assessment of biomarker panels in a cell sample and may be easily used for global and remote applications.

### TECHNICAL FIELD

The present disclosure relates generally to diagnostic and prognostic assays and a portable point of care system that performs such assays using an automated, artificial intelligence (AI) based molecular analyses of a subject sample. The system provides for rapid, cost-efficient multiplexable assessment of biomarker panels in a cell sample and may be easily used for global and remote applications.

### BACKGROUND

Efficient screening and early cancer detection programs are critical for prompt and effective treatment. However, in lower resource settings—such as rural areas lacking access to tertiary hospitals or under-resourced health facilities—it is not uncommon for biopsy diagnoses to take several weeks or months due to difficulties associated with specimen acquisition, logistics, and testing, and/or lack of medical personnel. Unfortunately, this may lead to delayed diagnosis, missed treatment options and increased mortality rates. Accordingly, the need exists for automated, rapid, portable, and affordable onsite diagnostic and profiling technologies.

### SUMMARY

Provided in accordance with the present disclosure are methods and devices for automated AI (artificial intelligence)-based molecular analyses of a sample from a subject.

In one aspect, an automated method of molecular sample analysis, for example cellular analysis, is provided. The automated method includes: (i) capturing an image, by an imaging device, the image including samples of interest tagged with a reporter(s), the image including at least one of a brightfield data, a darkfield data, a phase contrast data, a multichannel fluorescence data, or a diffraction data; (ii) communicating the captured image(s) to an image processing unit, the image processing unit including a first neural network; (iii) predicting, by the first neural network, sample features based on the captured image; (iv) predicting, by the first neural network, a segmentation based on the captured image; (v) communicating the sample features and the segmentation to one or more additional neural networks; (vi) predicting, by the one or more additional neural networks, a sample phenotype; (vii) determining a molecular readout or diagnosis/prognosis based on the sample phenotype; and (viii) displaying, on a display, the results. In a specific non-limiting embodiment, the molecular sample is a cellular sample, the sample feature is a cellular feature, and the sample phenotype is a cellular phenotype.

In another aspect, in a case where the captured image includes brightfield data, darkfield data, and/or phase contrast data, the method includes identifying the cellular features and determining a morphology of the identified cell. The cell features may include sub-cellular features, including for example, nuclei and mitochondria. In a case where the captured image includes multichannel fluorescence data, the first neural network further predicts a molecular phenotype. In a case where the captured image includes diffraction data, the first neural network predicts phase information. The method further includes generating 3D tomographic images based on the phase information and determining cell volume based on the generated 3D tomographic images.

In yet another aspect, a method is provided for molecular cellular analysis, the method including one or more of the following steps: (i) receiving a sample of a cellular specimen in a fixative; (ii) re-suspending the sample of the cellular specimen in a lyse buffer; (iii) tagging the sample of the cellular specimen with one or more reporters; (iv) immobilizing the sample of the cellular specimen on an optically transparent substrate(s); (v) capturing an image of the cellular specimen on the optically transparent substrate by an imaging device, the image including at least one of a brightfield data, a darkfield data, a phase contrast data, a fluorescence data, or a diffraction data; (vi) communicating the captured image to an image processing unit, the image processing unit including a first neural network; (vii) predicting, by the first neural network, cell features based on the captured image; (viii) predicting, by the first neural network, a segmentation based on the captured image; (ix) communicating the cell features and the segmentation to one or more additional neural networks; (x) predicting, by the one or more additional neural networks, a cellular phenotype; (xi) determining a molecular readout or diagnosis/prognosis based on the cellular phenotype; and (xii) displaying, on a display, the results. In an embodiment, the sample includes, for example, circulating blood cells, tissue cells, cells derived from bodily fluids, cells derived from a palpable mass, a visually detectable mass (e.g., the oral cavity), cells in a biopsy wash fluid, cells derived from an organ of interest or mass otherwise identified through diagnostic imaging and/or procedures. Such samples may be obtained by methods know to those of skill in the art including, for example, samples obtained by endoscopy, bronchoscopy, aspiration of a palpable mass; aspiration of a visually detectable mass; image guided aspiration by, for example, ultrasound or CT scan; endoscopic biopsy; surgical (i.e. incisional) fine needle aspiration (FNA); biopsy washes; thoracentesis, paracentesis, urine collection and mucosal brushing (e.g. cervical, buccal).

In one aspect, reporters include, for example, a lyophilized antibody, a chromogen, an affinity ligand, or combinations thereof. In an embodiment, the transparent substrate is glass, plastic, sapphire, and/or quartz.

With regards to fixation methods, such methods include, for example, those that (i) fix cells; (ii) lyse red blood cells; and/or (iii) preserve biomarkers, such as intracellular proteins (e.g., antigens) and nucleic acids, during sample transfer. Fixatives include for example, BD Lyse/Fix, alcohol and paraformaldehyde. In a specific embodiment, the formalin-based fixative solution CytoRich Red (CRR) is used. In one aspect, the fixation includes a fifteen-minute incubation in CRR. After fixation, cells may be permeabilized to enable the detection of biomarkers such as intracellular proteins and/or nucleic acids. Such permeabilization may be achieved using, for example, a saponin-based buffer.

In yet a further embodiment, a system for molecular analysis of a subject sample, including a cellular sample, is provided. The system includes: a re-suspension unit, a tagging unit, an immobilization unit, an imaging device, a display device, a processor, and a memory. The re-suspension unit is configured to re-suspend a sample of a cellular specimen in perm lyse buffer in a vial. The tagging unit is configured to tag a sample of the cellular specimen with a reporter. The immobilization unit is configured to immobilize the sample of the cellular specimen on an optically transparent substrate. The imaging device is configured to acquire images. The instructions, when executed cause the system to: receive the sample of a cellular specimen, the cellular specimen including a palpable or visual abnormality, a mass otherwise identified through diagnostic imaging or a needle sampled organ in a fixative; re-suspend the sample of the cellular specimen in perm lyse buffer in a vial; tag the sample of the cellular specimen with a reporter, the reporter including, for example, lyophilized antibody, chromogen, and/or affinity ligand combinations; immobilize the sample of the cellular specimen on an optically transparent substrate; and capture an image of the cellular specimen on the optically transparent substrate, by the imaging device. The image includes a brightfield data, a darkfield data, a phase contrast data, a fluorescence data, and/or a diffraction data. The instructions, when executed further cause the system to: communicate the captured image to an image processing unit, the image processing unit including a first neural network; predict, by the first neural network, cell features based on the captured image; predict, by the first neural network, a segmentation based on the captured image; communicate the cell features and the segmentation to additional neural networks; predict, by additional neural networks, a cellular phenotype; determine a molecular readout or diagnosis/prognosis based on the cellular phenotype; and display, on a display, the results.

The present disclosure provides an integrated molecular cytometer unit for performance of AI molecular and phenotypic diagnostic/prognosis methods, including cellular diagnostic/prognosis methods, provided herein. The cytometer unit includes (i) optical modules (ii) an imaging device; (iii) the mechanical components of the device; and (iv) a microcontroller (MCU) that operates each of the optical modules and the imaging device.

In an embodiment, the optical modules of the device can include a brightfield (or darkfield) module, which can be used to identify individual cells and measure their morphology. A phase-contrast module can be used to enable the identification of sub-cellular features, such as nuclei and mitochondria, for fine-grained cell classification. A fluorescent module can be used for molecular phenotyping based on, for example, immuno-staining of cells. A diffraction module can be used to retrieve phase information from cells, which is used to construct 3-dimensional tomographic images for cell-volume measurement. An on-board microcontroller unit (MCU) operates each module and an imaging device.

In an embodiment, kits are provided that contain all the necessary reagents, e.g., antibodies, buffers, and other reagents for carrying out the disclosed assays. In an embodiment, the kits are lyophilized thereby expanding the shelf life of the kits, allowing field testing and enabling storage in regular refrigerators rather than specialty freezers not commonly available in LMICs.

The imager of the device may include, for example, a CCD, CMOS, NMOS, or Quanta image sensor.

The mechanical components of the device can include, for example, a loading tray to receive a subject sample containing cells to be analyzed, a z-focus, and a means for lateral scanning.

Further, to the extent consistent, any of the aspects described herein may be used in conjunction with any or all the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic diagram of an exemplary cytometer system, in accordance with an aspect of the present disclosure;

FIG. 2 is a block diagram of an exemplary computing device forming part of the system of FIG. 1, in accordance with an aspect of the present disclosure;

FIG. 3 is a block diagram for the method of system control and analyzing results, in accordance with an aspect of the present disclosure;

FIG. 4 is a diagram of the automated data analysis, in accordance with an aspect of the present disclosure; and

FIGS. 5A-5B collectively present a flowchart of an exemplary method of molecular cellular analysis.

Further details and aspects of exemplary embodiments of the disclosure are described in more detail below with reference to the appended figures. Any of the above aspects and embodiments of the disclosure may be combined without departing from the scope of the disclosure.

### DETAILED DESCRIPTION

Aspects of the present disclosure are now described in detail with reference to the drawings in which like reference numerals designate identical or corresponding elements accordingly.

The present disclosure relates generally to methods and portable devices for performing molecular analyses of a subject sample. The methods and devices provided herein are particularly useful in point-of-care settings. More specifically, the present disclosure relates to AI-based molecular cellular analysis devices.

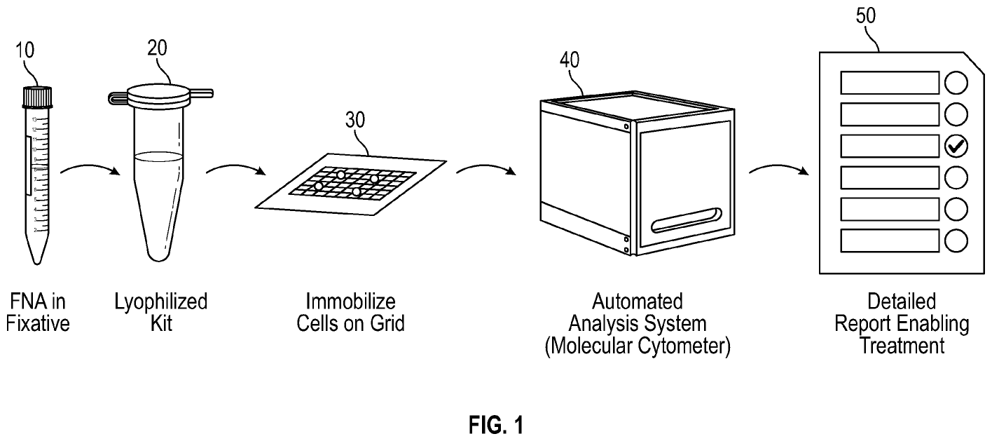

With reference to FIG. 1, there is shown a schematic diagram of an exemplary system 100, which may be used for processing digital images, according to an aspect of the present disclosure. This disclosure teaches the use of AI driven static cytometry technology to create a new class of portable, fast response profiling instrumentation for use in, for example, diagnostic and prognostic profiling. The disclosed methods and devices have applications in numerous bio-diagnostic and molecular profiling uses.

An advantage of the present system, over conventional microscopy, is its very low cost and ability to determine a diagnosis, or prognosis, without the intervention of a specialist physician. Furthermore, for cellular analysis, the system can interrogate a much larger number of cells from a single image acquisition, because it utilizes an exceptionally large field-of-view (FOV), one that is over 40 mm² in a simple optical configuration. In one embodiment, the system can image and computationally analyze >10⁴ individual cells in less than 30 sec. With such capabilities, the disclosed system surpasses flow cytometers and even skilled cytopathologists scanning across slides. By combining large FOV imaging with a trained neural network, the presently disclosed system can rapidly and automatically extract clinically relevant information with minimal human curation.

In embodiments, the cytometer unit and methods provided herein may be used to profile a sample, for example for diagnosis and/or prognosis (“diagnosis/prognosis”) of a number of diseases and disorders including but not limited to cancers, inflammatory and immunological disorders and diseases, organ toxicity and disease, including neurodegenerative, cardiovascular, renal, pulmonary, intestinal, and hematological diseases, to name a few. Also included, is the profiling, e.g., diagnosis/prognosis, of pathological conditions arising from infections with pathogens such as bacteria, fungi, viruses, and parasites. Specifically, a means for specifically detecting and identifying a virus, bacterium, or parasite within a sample of interest is provided.

Bacterial diseases include, but are not limited to, tuberculosis caused by Mycobacterium tuberculosis, pneumonia caused by bacteria such as Streptococcus and Pseudomonas, foodborne illnesses caused by bacteria such as E. coli, Shigella, and Salmonella, tetanus, typhoid fever, diphtheria, syphilis and leprosy. Viral diseases include, influenza, mumps, measles, chickenpox, ebola, HIV, rubella, hepatitis, COVID-19 and those caused by infection with CMV, herpesvirus and adenovirus to name a few. Pathogenic parasites include Plasmodium that causes malaria, Entamoeba that causes amoebiasis, Giardia that causes giardiasis, Trypanosoma brucei that causes African trypanosomiasis, Toxoplasma gondii that causes toxoplasmosis, Leishmania that causes leishmaniasis, Ascaris lumbricoides that causes ascariasis and Schistosoma that causes schistosomiasis.

In a specific embodiment, the cytometer unit and methods provided herein may be used to quickly profile a sample for diagnosis and/or prognosis of COVID-19 disease. In such instances, complications associated with COVID-19 may be detected. Such complications include, for example, development of inflammatory diseases, infectious diseases, drug toxicities, organ failure (e.g., liver), parenchymal organ toxicity, hepatocyte necrosis, glomerialar cell necrosis/apoptosis, cell death, and coagulopathies. In one aspect, immune cell profiling using immune cell markers may be done to assess the inflammatory response from Covid-19 complications in a patient. With regard to detection of coagulopathies, the present disclosure provides through the use of FNA as a means for not interrupting anti-coagulation. Based on the detection and determination of the severity of such complications, drug treatment regiments may be designed for efficient treatment of patients infected with the virus.

In one specific aspect, the cytometer unit and diagnostic/prognostic methods disclosed herein, are used to diagnose and/or prognose cancer within a subject. The cytometer unit may be used to differentiate between high- and low-grade malignancy subtypes based on biomarker expression. Cancers to be diagnosed/prognosed include any type of solid tumors, such as cancer of the skin, head, neck, thyroid, lungs, breast, pancreas, colon, stomach, prostate, ovary, liver, kidney, intestine, and other organs and glands. The cancer may also be a blood or lymphatic cancer, which is generally understood as any kind of hematological malignancy (e.g., lymphoma, leukemia, circulating tumor cells of solid tumors).

The profiling of a sample, for example, for diagnosis/prognosis of a disease or disorder, is based on the detection of a panel, e.g. one or more, of different biomarkers present within a subject sample of interest, the detection of said biomarkers being known to be associated with a known disease, disorder or condition. Detection of such biomarkers may indicate the presence of a certain cell type within a sample, for example a cancer cell type, or the presence of cellular constituents such as proteins, nucleic acids, biologically active compounds or other cellular components that are indicative of and associated with the presence of a known disease or disorder. Such biomarkers may further include general cell morphology, as well as distinctive sub-cellular features such as nuclei and mitochondria, for fine grained cell classification.

A wide range of different cancer-associated and host cell biomarkers are well known in the art, but few alone are ever diagnostic with the certainty required for therapeutic intervention. Accordingly, it is advantageous to use combinations of multiple biomarkers to more accurately identify and quantitate the presence of certain cell types in a sample and/or to diagnose or prognose a disease or disorder within a subject. For example, numerous biomarkers have been identified that are expressed on different cancer cells or subtypes of immune cells thereby providing a means for identification of said types of cells.

Biomarkers, the presence of which is associated with certain cell types, such as specific tissue cells, cancer cells, or immune system cells, are well known to those skilled in the art. Additionally, biomarkers whose presence is associated with a pathogenic condition, such as bacterial, viral or parasitic expressed proteins, are also well known to those skilled in the art and may be used in the practice of the methods disclosed herein.

The biomarkers chosen for any given assay may depend on the specific disease, disorder or condition which a test subject is suspected of having. Such biomarkers may include, for example, proteins, nucleic acids, and biologically active compounds found within a subject sample, the presence of which is associated with a specific disease, disorder or condition.

In non-limiting embodiments, biomarkers which may be used either individually or in combination, include, for example, epithelial cancer cell markers including, for example, EpCAM, HER2, ER, PR, Ki67, EGFR, CD24, Lin8a, GPA22, CD133, MET, ALK, MUC1, MUC5ac, TTF-1, CYFRA 21-1, WNT2, CYFRA 21-1 Trop2, CD44, and p16 among others. Breast cancer associated cell markers include, for example, HER2, ER, PR, and Ki67. Colon cancer associated markers include, for example, EpCAM, EGFR, CD24, GPA33, and CD133. Lung cancer associated markers include, for example, EGFR, MET, ALK, MUC1, HER2, TTF-1, and CYFRA 21-1. Lymphoma associated markers include, for example, k (kappa), λ (lambda), CD 19/20, and Ki67. BRAF has been shown to be associated with melanoma. HNSCC associated markers included P16, p40, p63, Quad marker (EPCAM, EGFR, MUc1), tsMHC1, tsMHC2. Gastrointestinal associated markers include EpCAM, EGFR, CD24, GPA33, and CD133. HCC liver cancer markers include GPC3, HepPar-1, CEA, AFP, Arg-1. Immune cell markers include CD45, CD1a CD3, CD4, CD8, CD11B, CD11C, CD20, CD45, CD45RA, CD45RO, CD49a, CD66B, CD68, CD103, CD161, CD163, FoxP3, PD-L1, PD1, TCF1, GZMB, IFNg, MHCI, MHCII, IL12b, TAM (Tyro3, Axl, and Mer) family of receptors, and TAN (tumor-associated neutrophil) among others.

The assays disclosed herein provide for improved cell isolation and high-resolution characterization of expression patterns of biomarkers resulting in enhanced diagnostic and prognostic value. In contrast to typical cytopathology assays performed in a clinical laboratory setting based on the use of color or immunohistochemical stains and detection of one or two markers, the present disclosure provides a method for simultaneously assaying (i) a panel of multiple biomarkers; through the use of (ii) multiple detection means. The disclosed method provides increased sensitivity and accuracy for diagnostic and prognostic of diseases, disorders, or conditions and can easily be performed using the described portable device in a laboratory or point-of-care setting.

The presence of certain biomarkers may be used by physicians to design patient-specific treatment protocols that are most likely to result in disease treatments, including, for example, regression of the cancer state or relief from pathogenic infections. Specifically, the methods include correlating diagnostic or prognostic results, e.g., detection of biomarkers indicating the presence and quantity of specific cell types within a sample, or expression of specific proteins, or RNA, or DNA (including mutated variants) within a sample, or presence of biologically active compounds, with disease severity or to other clinical parameters such as predicted response to treatment and overall survival. In a specific embodiment, the methods include correlating the diagnostic/prognostic results to the therapeutic outcome of cancer treatment.

In addition to detection of relevant cancer-associated genes and gene products, the cytometry unit disclosed herein may be used to detect clinical biomarkers that may be associated with the likelihood of successful immunotherapy. Such detection protocols may be used by physicians to better identify patients who may be more likely to benefit from immunotherapy. Such markers include numbers and activity profiles of the following cells: CD45, CD1a CD3, CD4, CD8, CD11B, CD11C, CD20, CD45, CD45RA, CD45RO, CD49a, CD66B, CD68, CD103, CD161, CD163, FoxP3, PD-L1, PD1, TCF1, GZMB, IFNg, MHCI, MHCII, IL12b, TAM (Tyro3, Axl, and Mer) family of receptors, and TAN (tumor-associated neutrophil) among others.

In addition, the cytometry unit disclosed herein may be used to detect clinical biomarkers that may be associated with a bacterial, viral or parasitic infection. Detection protocols may be used by physicians to better identify patients who are infected with such pathogens. Such markers include bacterial, viral or parasitic nucleic acids and/or protein products. Such biomarkers, for detection of pathogenic infections, may also include anti-pathogen circulating antibodies within a suspect infected subject.

In a specific embodiment, biomarkers associated with COVID-19 may be detected. Such biomarkers include, for example, COVID-19 nucleic acids as well as viral encoded proteins/polypeptides, e.g., the viral spike, membrane and/or envelope protein. In one aspect, anti-COVID-19 antibodies may be detected.

Detection of said biomarkers within a sample may be achieved through use of reagents that recognize and bind to said biomarkers (target biomarkers), e.g., cell surface-expressed proteins, intracellular proteins, nucleic acids, viruses, bacteria, and biologically active compounds, etc. The reagents may be tagged with labeling agents that generate a distinct optical signature that is detected by the cytometer device upon binding to its cognate biomarker.

Reagents include, for example, affinity ligands such as antibodies, aptamers, peptides, and complementary nucleic acid molecules that bind to the biomarker of interest. In one embodiment, complementary nucleic acids that target binding to a specific DNA or RNA sequence may be used as an affinity ligand. In such instances, the complementary nucleic acid affinity ligand may be labeled with a detection molecule such as biotin, a fluorophore or enzyme. In another aspect, the complementary nucleic acid may be a bacterial, viral or parasitic nucleic acid. The choice of such pathogen-associated complementary nucleic acids, for use as a biomarker, is well known to those of skill in the art.

In a specific embodiment, the affinity reagent is an antibody molecule. “Antibody molecule” as used herein is intended to include intact antibodies, such as polyclonal antibodies or monoclonal antibodies (mAbs), as well as proteolytic fragments thereof such as the Fab or F(ab′)₂ fragments, chimeric antibodies, nanobodies, recombinant and engineered antibodies, and fragments thereof, as well as other molecules having at least an antigen-binding site of an antibody. Typically, and advantageously, the antibodies are labeled, or tagged, directly (conjugated), or indirectly, to a labeling agent such as a fluorophore or chromophore. In an embodiment, a cocktail of antibodies for primary and secondary immunostaining may be used to reduce assay time. In one aspect, the antibodies include lyophilized antibody cocktails which optimize for storage of the antibodies.

In one aspect, the affinity reagent is detected indirectly through the use of a labeled reagent that binds to the affinity reagent. Labeling agents that may be used to tag the biomarker binding reagents, directly or indirectly, including, for example, chromophores, fluorochromes, DNA barcodes, nanoparticles, and enzymes. Labels may include, for example, a fluorescent label, a chemiluminescent label, and electro-chemiluminescent label. Examples of labeling substances include, but are not limited to, fluorescent substances such as green fluorescent protein, red fluorescent protein, 4′,6-diamidino-2′-phenylindole dihydrochloride (DAPI), Hoechst 33342, BIODIPY, indocyanines, atto dyes, fluorescein isothiocyanate, tetramethyl rhodamine isothiocyanate, substituted rhodamine isothiocyanate, dichlorotriazine isothiocyanate, Alexa or AlexaFluoro, and the like.

The sample preparatory steps involved depend on the type of analysis planned. Typically, the disclosed assays will utilize biological samples or isolates from the subject to be tested (“subject sample”). Such samples may include needle rinses and aspirates, digested tissues, blood, plasma, spinal fluid, surgical, thoracentesis, paracentesis samples, fluid aspirations, stool, and/or urine. Samples mean tissue or bodily fluid, such as blood or plasma, that is drawn from a subject and from which the concentrations or levels of diagnostically informative biomarkers may be determined. Methods for collection of biological samples for use in diagnostic methods are well known to those of skill in the art. In an embodiment, such samples may be obtained by fine needle aspiration (FNA). In a typical clinical setting, each single needle pass (20-22 gauge) provides about 10² to 10⁵ cells, depending on technique and type of lesion.

In addition to human medical diagnostics, the cytometric unit and methods disclosed herein can be used to detect the constituents of a wide range of different samples, including, for example, veterinarian, agricultural, wildlife, fish, and aquatic animal samples. Environmental samples, may include, for example, aquatic samples derived from streams, lakes, the ocean ponds, and pools. Foods and beverages may also be assayed. Such samples can be used to diagnose and prognose diseases and disorders in animals other than humans or to detect the presence of pathogenic contaminants or toxins in food and water samples.

In one aspect, cells within a collected sample are captured and stained within a disposable fluidic cartridge that contains the reagents for detection of one or more of the biomarkers of choice. On one side of the cartridge's bottom, optically transparent surface may be functionalized with reagents for capture of specific cell types and/or detection of biomarkers of interest. Still further, the cells may be mounted, which involves attaching the samples to a substrate for observation and analysis.

In various embodiments, the cytometer unit includes bright-field, phase-contrast, fluorescent, or holographic optical modules integrated in a miniaturized format for improved diagnosis and/or prognosis of diseases and disorders, including the monitoring of tumors and other lesions involving immune cell infiltration. The cytometer unit provides a high-resolution quantitative means for disease prognostic and therapy based on the detection of predictive biomarkers. Thus, the cytometer unit and methods described herein surprisingly provide for highly accurate and informative separation of cell populations as well as detection of biomarkers expressed within the separated cell populations.

In various embodiments, cellular samples may be directly processed through a lyophilized (freeze-dried) kit containing all reagents required for analysis. In various embodiments, when the analyte is added, cells become ready for detection. In various embodiments, multiple assay panels may be created in separate chambers or wells associated with each assay within the disposable fluidic cartridge. In various embodiments, multiple measurements and auto-calibration may be provided to add increased accuracy and precision to the measurement. In various embodiments, a signal processing architecture tailored to the optical measurement may provide both time domain and frequency domain processing and analysis.

With continued reference to FIG. 1, a schematic of the major subsystems of the basic cytometer system is shown. The sample 10 consists of a cellular specimen, for example, a fine needle aspirate of a palpable mass in a fixative. The cell pellet derived from 10 is resuspended in perm lyse buffer in a vial 20 containing lyophilized antibody, chromogen and/or affinity ligand combinations and cryoprotectant. The cell pellet from 20 is immobilized on an optically transparent substrate 30 using covalent bonding, a chemical glue or technical device. The sample 30 is then inserted into the molecular cytometer 40, where multiplexed measurements are performed in an automated and calibrated fashion. Data from 40 is analyzed using neural networks (a type of AI), and results are displayed in a report 50. The latter includes a summary diagnosis/prognosis for medical and therapeutic decision making.

The molecular cytometer 40 may consist of an imaging component (e.g., an LED and a CMOS image sensor), an autofocus device, a microcomputer (e.g., Raspberry Pi 3 with a wireless and Bluetooth unit) and may include a touchscreen. In one aspect, the components of the cytometer are designed for limited mobility. The case may be fabricated using three-dimensional printing. In a non-limiting embodiment, the overall size may be approximately 205 mm (L)×120 mm (W)×175 mm (H). The weight may be approximately 1.4 kg. The molecular cytometer 40 may be powered by either a corded power supply adaptor, or a lithium or a lithium-ion battery back, or any other suitable power supply.

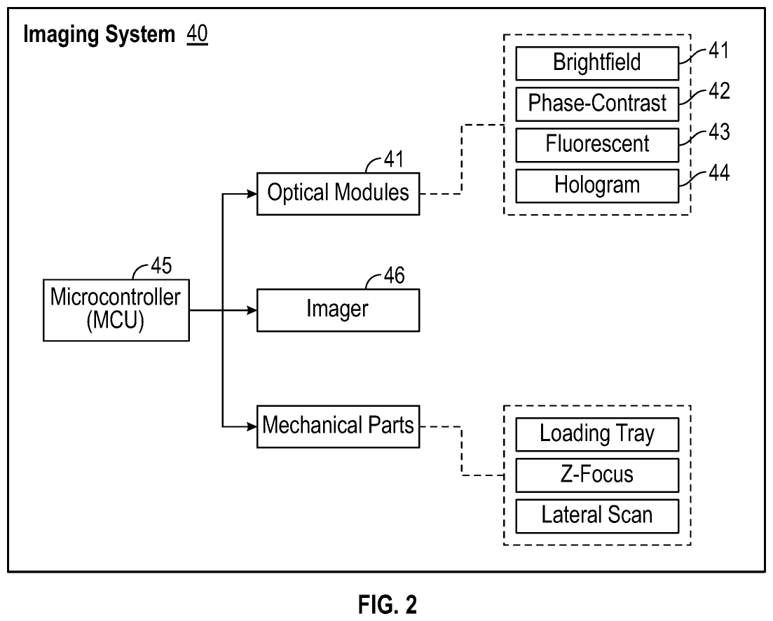

With reference to FIG. 2, a diagram of the molecular cytometer 40 is shown. The molecular cytometer 40 is equipped with an array 41 of optical modules, each yielding unique information of cell phenotypes. The brightfield (or darkfield) module 42 is used to identify individual cells and measure their morphology. The phase-contrast module 42 enables the identification of sub-cellular features, such as nuclei and mitochondria, for fine-grained cell classification. The fluorescent module 43 allows for molecular phenotyping based on, but not limited to, immuno-staining of cells. The diffraction module 44 retrieves the phase information from cells, which is used to construct 3-dimensional tomographic images for cell-volume measurement. An on-board microcontroller unit (MCU) 45 operates each module and an imaging device 46. In various embodiments, an LED may be used as an excitation source and a GRIN lens as an objective.

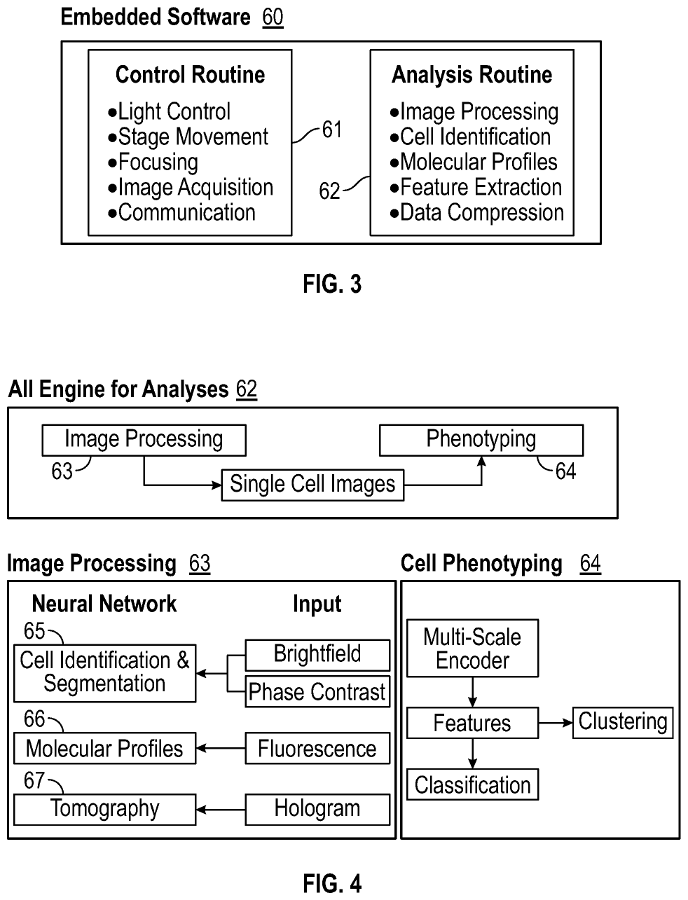

With reference to FIG. 3, a diagram of the software 60 installed in the MCU 45 to control the system and analyze results is shown. The control routine 61 synchronizes the operation of each optical modules 41, 42, 43, 44, and the imaging device 46. The AI engine 62 contains pre-trained neural networks and other auxiliary subroutines to enable on-spot image processing.

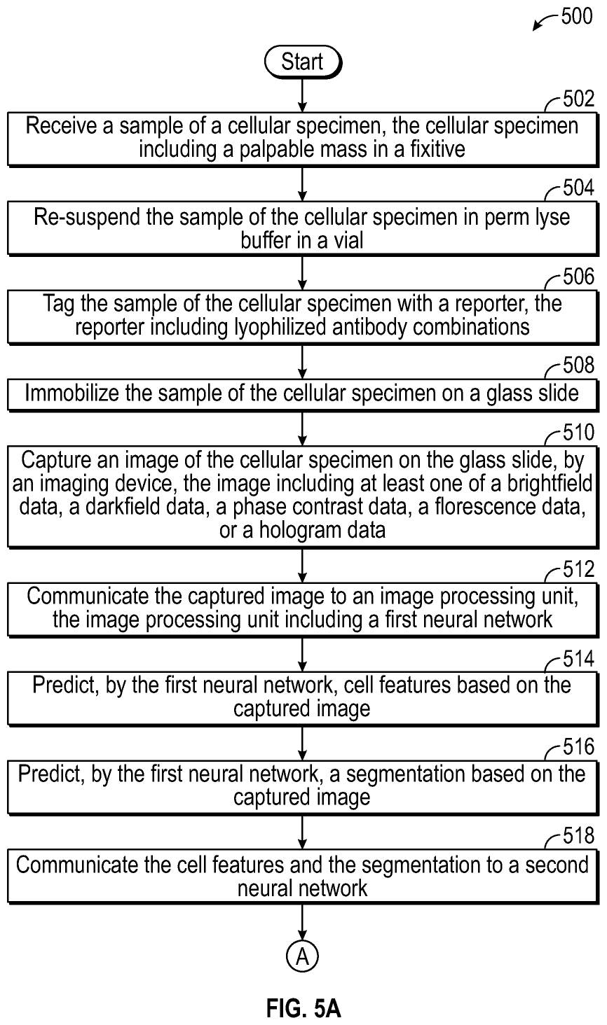

With reference to FIG. 4, a diagram of the automated data analysis is shown. The AI engine 62 can have multiple modules. In one example, it has two submodules, one for image processing 63 and the other for cell phenotyping 64. The image processing unit has pre-trained neural networks for cell identification and segmentation 65 from brightfield and phase contrast images. For each segmented region, the profiling unit 66 assigns molecular information based on immunostaining results. A separate neural network 67 renders 3-dimensional tomographic images from diffraction patterns and calculate cell volumes. Following the image processing, the analysis routine 64 takes cellular and subcellular features as an input, and deconvolve them for cellular phenotyping.

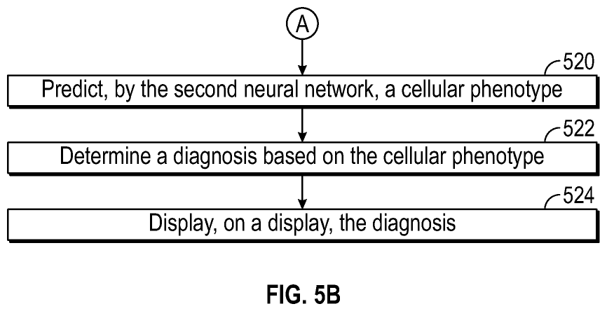

With reference to FIGS. 5A and 5B, there is shown a flowchart of an exemplary method 500 of molecular cellular analysis. While the below-described steps of method 500 are described in an exemplary sequence or order of operations, those skilled in the art will recognize that some or all of these steps may be performed in a different sequence or order of operations, or may be duplicated or omitted, without departing from the scope of the present disclosure.

Initially, at step 502, the system receives a sample of a cellular specimen 10. For example, a fine needle aspirate of a palpable mass in a fixative.

At step 504, the system re-suspends the sample of the cellular specimen 10 in perm lyse buffer in a vial 20. In various embodiments, the perm/lyse buffer in the vial 20 may contain lyophilized antibody, chromogen, and/or affinity ligand combinations and cryoprotectant.

At step 506, the system tags the sample of the cellular specimen 10 with a reporter. The reporter includes lyophilized antibody, chromogen, and/or affinity ligand combinations. In various embodiments, the cellular specimen 10 may be stained with unique chromogens (e.g., hematoxylin/eosin, others) and specific antibodies, and consensus reads are performed. At step 508, the system immobilizes the sample of the cellular specimen 10 on an optically transparent substrate 30. It is contemplated that the system includes multiple assay panels in separate chambers or wells and channels associated with each assay.

At step 510, the system captures an image of the cellular specimen 10 on an optically transparent substrate 30, by an imaging device. The image includes brightfield data, darkfield data, phase contrast data, multichannel fluorescence data, and/or diffraction data. In brightfield data, sample illumination is transmitted (i.e., illuminated from below and observed from above) white light, and contrast in the sample is caused by attenuation of the transmitted light in dense areas of the sample. Dark-field microscopy (also called dark-ground microscopy) describes microscopy methods, in both light and electron microscopy, which exclude the unscattered beam from the image. As a result, the field around the specimen (i.e., where there is no specimen to scatter the beam) is generally dark. Phase-contrast microscopy is an optical microscopy technique that converts phase shifts in light passing through a transparent specimen to brightness changes in the image. Phase shifts themselves are invisible but become visible when shown as brightness variations. In various embodiments, the imaging device may include at least one of a CCD, CMOS, NMOS, or Quanta image sensor. In various embodiments, the system may include a multi-band pass filter, e.g., an optics configuration for multichannel cellular analysis.

In various embodiments, the signal processing may include several modes of operation to capture the effects of the optical measurements, including both time and frequency domain transforms and analysis. In various embodiments, the image may include 2 or 3-dimensional data by combining multiples images taken in different angles.

At step 512, the system communicates the captured image to an image processing unit. The image processing unit includes a first neural network. In various embodiments, the captured image may be processed remotely over a wired or a wireless network. At step 514, the image processing unit predicts, by the first neural network, cell features based on the captured image. At step 516, the image processing unit predicts, by the first neural network, a segmentation based on the captured image. In various embodiments, the neural network includes a convolutional neural network (CNN). In various embodiments, a convolution layer is followed by an activation extracts feature. In various embodiments, the activation function is a rectified linear unit (ReLU). In various embodiments, pooling may be used to downsample intermediate layers. In various embodiments, 2D feature maps may be flattened for producing a probability distribution. The CNN then provides a classification of the images. In various embodiments, in a case where the captured image includes at least one of the brightfield data or the darkfield data: the cell features include a cell identification and the system further includes determining a morphology of the identified cell. In various embodiments, in a case where the captured image includes the phase contrast data: the cell features include sub-cellular features including nuclei and mitochondria. In various embodiments, in a case where the captured image includes fluorescence data: the predicting, by the first neural network, further includes a molecular phenotype. Fluorescent immunophenotyping uses fluorescently conjugated antibodies to identify, characterize, and quantify distinct subpopulations of cells within heterogeneous single-cell populations, either in the context of tissue or in a single-cell suspension. In various embodiments, in a case where the captured image includes the diffraction data: the system may predict, by the first neural network, phase information. The system may generate, based on the phase information, 3D tomographic images, and determine cell volume based on the generated 3D tomographic images.

Machine learning algorithms are advantageous for use in predicting medical diagnosis/prognosis at least in that machine learning algorithms may improve the functionality of complex assay diagnosis/prognosis. Machine learning algorithms utilize the initial input data, e.g., image data, to determine statistical features and/or correlations that enable the identification of cell phenotyping by analyzing data therefrom. Thus, with one or more machine learning algorithms having been trained as detailed above, such can be used to identify cell phenotypes. More specifically, a processor 45 is configured, in response to receiving sensed data from optical modules, to input the sensed data into the machine learning algorithm(s) stored in memory in order to correctly identify cell features. Although described with respect to molecular cytometric system, the aspects and features of the processor 45 and the machine learning algorithms configured for use therewith are equally applicable for use with other medical analysis or diagnosis/prognosis systems.

The terms “artificial intelligence (AI),” “data models,” “deep learning,” or “machine learning” may include, but are not limited to, neural networks, deep neural networks, convolutional neural networks (CNN), recurrent neural networks (RNN), generative adversarial networks (GAN), Bayesian Regression, Naive Bayes, Monte Carlo Methods, nearest neighbors, least squares, means, and support vector regression, among other data science and artificial science techniques. Exemplary uses are identifying patterns and making predictions relating to static cytometry, which will be described in more detail hereinbelow.

The term “application” may include a computer program designed to perform particular functions, tasks, or activities for the benefit of a user. Application may refer to, for example, software running locally or remotely, as a standalone program or in a web browser, or other software which would be understood by one skilled in the art to be an application. An application may run on the processor 45 or on a user device, including for example, on a mobile device, an IoT device, or a server system.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), graphics processing unit (GPU) or the like. The controller may also include a memory to store data and/or instructions that, when executed by the one or more processors, causes the one or more processors to perform one or more methods and/or algorithms.

At step 518, the system communicates the cell features and the segmentation to one or more additional neural networks. At step 520, the system predicts a cellular phenotype based on output of the one or more additional neural networks. In various embodiments, the one or more additional neural networks may include a CNN.

At step 522, the system determines a molecular readout or diagnosis/prognosis based on the cellular phenotype. In various embodiments, the one or more additional neural networks may include a pre-trained neural network. It is contemplated that the first neural network and the one or more additional neural networks may be trained remotely using a separate processor. In various embodiments, the training of the first neural network and the one or more additional neural networks may include data augmentation. For example, the images may be stretched, transformed, pixel-shifted, rotated, and/or mirrored.

At step 524, the system displays, on a display, the result. For example, the resulting molecular readout or diagnosis/prognosis may provide entire molecular signatures corresponding to cancer sub-types in multiplexed fashion for high-throughput, cellular analysis.

In various embodiments, the system is capable of self-calibration. For example, the system may measure the intensities of all light sources and adjust operation settings (e.g., power to a light source, amplification gain in the detector, and image acquisition time) to ensure consistent imaging. In various embodiments, the system may initialize mechanical movements (e.g., auto z-focusing, lateral stage translation). It can also find the orientation of sample slides (e.g., skew angle) and automatically correct the stage movements for sample scanning.

In various embodiments, the neural network may be pre-trained. A training set may consist of labeled Z-stacks of transmitted and fluorescent micrographs. The input images may include brightfield images, darkfield images, phase contrast images, multichannel fluorescence images, and/or diffraction patterns. For example, the images may be stained for subfeatures of a cell such as a nucleus or a neuron and labeled accordingly. The training may include supervised learning or unsupervised learning.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, MATLAB, Julia, Python, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms, or codes may be contained on one or more machine-readable media or memory. The term “memory” may include a mechanism that provides (for example, stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. A method of automated, artificial intelligence (AI)-based molecular diagnostic analysis of a subject sample, the method comprising:
capturing multi-modal images, by an imaging device, the images including a subject sample tagged with reporters, the image types including at least one of a brightfield data, a darkfield data, a phase contrast data, multichannel fluorescence data, or diffraction data;
communicating the captured image to an image processing unit, the image processing unit including a first neural network;
predicting, by the first neural network, cell features based on the captured image;
predicting, by the first neural network, a segmentation based on the captured image;
communicating the cell features and the segmentation to one or more additional neural networks;
predicting, by the one or more additional neural networks, a cellular phenotype;
determining at least one of a molecular readout or diagnosis/prognosis based on the cellular phenotype; and
displaying, on a display, at least one of the readout or the diagnosis/prognosis.

2. The method of claim 1, wherein the method further includes:
in a case where the captured image includes at least one of the brightfield data, the darkfield data, or the phase contrast data:
the cell features include a cell identification;
the method further includes determining a morphology of the identified cell; and
the cell features include sub-cellular features, the sub cellular features including nuclei and mitochondria;
in a case where the captured image includes multichannel fluorescence data:
the predicting, by the first neural network, further includes a molecular phenotype; and
in a case where the captured image includes the diffraction data:
predicting, by the first neural network, phase information;
generating, based on the phase information, 3D tomographic images; and
determining cell volume based on the generated 3D tomographic images.

3. The method of claim 1 or 2, wherein the subject sample is a cellular sample.

4. The method of claim 1 or 2, wherein the reporter detects the presence of one or more biomarkers associated with a disease or disorder within the sample.

5. The method of claim 4, wherein the biomarker is selected from the group consisting of EpCAM, HER2, ER, PR, Ki67, EGFR, CD24, Lin8a, GPA22, CD133, MET, ALK, MUC1, MUC5ac, TTF-1, CYFRA 21-1, WNT2, CYFRA 21-1 Trop2, CD44, p16, GPA33, EpCA, BRAFF, EGFR, MET, k (kappa), λ (lambda), CD 19/20, p40, p63, tsMHC1, tsMHC2, CD133, GPC3, HepPar-1, CEA, AFP, Arg-1, CD45, CD1a CD3, CD4, CD8, CD11B, CD11C, CD20, CD45, CD45RA, CD45RO, CD49a, CD66B, CD68, CD103, CD161, CD163, FoxP3, PD-L1, PD1, TCF1, GZMB, IFNg, MHCI, MHCII, IL12b, the TAM (Tyro3, Axl, and Mer) family of receptors and TAN.

6. The method of claim 4, wherein the disease is cancer.

7. The method of claim 6, wherein the cancer is cancer of the skin, head, neck, thyroid, lungs, breast, pancreas, colon, stomach, prostate, ovary, liver, kidney, intestine, glands, blood, lymphatic cancer and hematological malignancies such as lymphoma and leukemia.

8. The method of claim 4, wherein the disease results from infection with a pathogen.

9. The method of claim 8, wherein the pathogen is selected from the group consisting of a bacterial, viral, and parasitic pathogen.

10. The method of claim 9, wherein the pathogen is COVID-19.

11. The method of claim 4, wherein the disorder is an immunological disorder.

12. The method of claim 1 or 2, wherein the reporter is an antibody, chromogen, microsphere, nanoparticle, a molecule affinity ligand or a nucleic acid.

13. The method of claim 12, wherein the reporter is directly tagged with a detectable label.

14. The method of claim 12, wherein the reporter is tagged indirectly with a detectable label.

15. The method of claim 1 or 2, wherein the subject sample is obtained by endoscopy, bronchoscopy, aspiration of a palpable mass; aspiration of a visually detectable mass; image guided aspiration by, for example, ultrasound or CT scan; endoscopic biopsy; surgical (i.e. incisional) fine needle aspiration (FNA); biopsy washes; thoracentesis, paracentesis, urine collection and mucosal brushing (e.g. cervical, buccal).

16. A method for molecular cellular analysis, the method comprising:
receiving a sample of a cellular specimen,
re-suspending the sample of the cellular specimen in perm lyse buffer in a vial;
tagging the sample of the cellular specimen with a reporter;
immobilizing the sample of the cellular specimen on an optically transparent substrate;
capturing an image of the cellular specimen on the optically transparent substrate, by an imaging device, the image including at least one of a brightfield data, a darkfield data, a phase contrast data, a fluorescence data, or a diffraction data;
communicating the captured image to an image processing unit, the image processing unit including a first neural network;
predicting, by the first neural network, cell features based on the captured image;
predicting, by the first neural network, a segmentation based on the captured image;
communicating the cell features and the segmentation to one or more additional neural networks;
predicting, by the one or more additional neural networks, a cellular phenotype;
determining a molecular readout or diagnosis/prognosis based on the cellular phenotype; and
displaying, on a display, the readout or diagnosis/prognosis.

17. The method of claim 16, wherein the cellular specimen is obtained by endoscopy, bronchoscopy, aspiration of a palpable mass; aspiration of a visually detectable mass; image guided aspiration by, for example, ultrasound or CT scan; endoscopic biopsy; surgical (i.e. incisional) fine needle aspiration (FNA); biopsy washes; thoracentesis, paracentesis, urine collection and mucosal brushing (e.g. cervical, buccal).

18. The method of claim 16, wherein the reporter detects the presence of one or more biomarkers associated with a disease or disorder within the sample.

19. The method of claim 18, wherein the biomarker is selected from the group consisting of EpCAM, HER2, ER, PR, Ki67, EGFR, CD24, Lin8a, GPA22, CD133, MET, ALK, MUC1, MUC5ac, TTF-1, CYFRA 21-1, WNT2, CYFRA 21-1 Trop2, CD44, p16, EpCA, BRAFF, GPA33, EGFR, MET, k (kappa), λ (lambda), CD 19/20, p40, p63, tsMHC1, tsMHC2, CD133, GPC3, HepPar-1, CEA, AFP, Arg-1, CD45, CD1a CD3, CD4, CD8, CD11B, CD11C, CD20, CD45, CD45RA, CD45RO, CD49a, CD66B, CD68, CD103, CD161, CD163, FoxP3, PD-L1, PD1, TCF1, GZMB, IFNg, MHCI, MHCII, IL12b, the TAM (Tyro3, Axl, and Mer) family of receptors and TAN.

20. The method of claim 18, wherein the disease is cancer.

21. The method of claim 20, wherein the cancer is selected from the group consisting of cancer of the skin, head, neck, thyroid, lungs, breast, pancreas, colon, stomach, prostate, ovary, liver, kidney, intestine, glands, blood, lymphatic cancer and hematological malignancies such as lymphoma and leukemia.

22. The method of claim 18, wherein the disease results from infection with a pathogen.

23. The method of claim 22, wherein the pathogen is selected from the group consisting of a bacterial, viral, and parasitic pathogen.

24. The method of claim 23, wherein the viral pathogen is COVID-19.

25. The method of claim 18, wherein the disorder is an immunological disorder.

26. The method of claim 16, wherein the reporter is an antibody, chromogen, microsphere, nanoparticle, a molecule affinity ligand or a nucleic acid.

27. The method of claim 26, wherein the reporter is directly tagged with a detectable label.

28. The method of claim 26, wherein the reporter is tagged indirectly with a detectable label.

29. A system for molecular and phenotypic cellular analyses, the system comprising:
a re-suspension unit configured to re-suspend a sample of a cellular specimen in perm lyse buffer in a vial;
a tagging unit configured to tag a sample of the cellular specimen with a reporter;
an immobilization unit configured to immobilize the sample of the cellular specimen on an optically transparent substrate;
an imaging device configured to acquire images;
a display device;
a processor; and
a memory, including instructions thereon, which when executed cause the system to:
receive the sample of a cellular specimen;
re-suspend the sample of the cellular specimen in perm lyse buffer in a vial;
tag the sample of the cellular specimen with a reporter;
immobilize the sample of the cellular specimen on an optically transparent substrate;
capture an image of the cellular specimen on the optically transparent substrate, by the imaging device, the image including at least one of a brightfield data, a darkfield data, a phase contrast data, a multichannel fluorescence data, or a diffraction data;
communicate the captured image to an image processing unit, the image processing unit including a first neural network;
predict, by the first neural network, cell features based on the captured image;
predict, by the first neural network, a segmentation based on the captured image;
communicate the cell features and the segmentation to one or more additional neural networks;
predict, by the one or more additional neural networks, a cellular phenotype;
determine a diagnosis/prognosis based on the cellular phenotype; and
display, on the display, the result.

30. The system of claim 29, wherein the cellular specimen is obtained by endoscopy, bronchoscopy, aspiration of a palpable mass; aspiration of a visually detectable mass; image guided aspiration by, for example, ultrasound or CT scan; endoscopic biopsy; surgical (i.e. incisional) fine needle aspiration (FNA); biopsy washes; thoracentesis, paracentesis, urine collection and mucosal brushing (e.g. cervical, buccal).

31. The system of claim 29, wherein the reporter detects the presence of one or more biomarkers associated with a disease or disorder within the sample.

32. The system of claim 31, wherein the biomarker is selected from the group consisting of EpCAM, HER2, ER, PR, Ki67, EGFR, CD24, Lin8a, GPA22, CD133, MET, ALK, MUC1, MUC5ac, TTF-1, CYFRA 21-1, WNT2, CYFRA 21-1 Trop2, CD44, p16, EpCA, BRAFF, GPA33, EGFR, MET, k (kappa), λ (lambda), CD 19/20, p40, p63, tsMHC1, tsMHC2, CD133, GPC3, HepPar-1, CEA, AFP, Arg-1, CD45, CD1a CD3, CD4, CD8, CD11B, CD11C, CD20, CD45, CD45RA, CD45RO, CD49a, CD66B, CD68, CD103, CD161, CD163, FoxP3, PD-L1, PD1, TCF1, GZMB, IFNg, MHCI, MHCII, IL12b, the TAM (Tyro3, Axl, and Mer) family of receptors and TAN.

33. The system of claim 31, wherein the disease is cancer.

34. The system of claim 33, wherein the cancer is cancer of the skin, head, neck, thyroid, lungs, breast, pancreas, colon, stomach, prostate, ovary, liver, kidney, intestine, glands, blood , lymphatic cancer, hematological malignancies such as lymphoma and leukemia.

35. The system of claim 31, wherein the disease results from infection with a pathogen.

36. The system of claim 35, wherein the pathogen is selected from the group consisting of a bacterial, viral, and parasitic pathogen.

37. The system of claim 36, wherein the viral pathogen is COVID-19.

38. The system of claim 31, wherein the disease is an immunological disorder.

39. The system of claim 29, wherein the reporter is an antibody, chromogen, microsphere, nanoparticle, a molecule affinity ligand or a nucleic acid.

40. The system of claim 39, wherein the reporter is directly tagged with a detectable label.

41. The system of claim 39, wherein the reporter is tagged indirectly with a detectable label.
